(19) United States
(12) Patent Application Publication
(10) Pub. No.: US 2010/0261962 A1
(43) Pub. Date: Oct. 14, 2010

(54) NATURAL ORIFICE TRANSLUMINAL ENDOSCOPIC SURGERY OVERTUBE AND METHOD OF INTRODUCING MULTIPLE ENDOSCOPES

(76) Inventor: Joseph S. Friedberg, Philadelphia, PA (US)

Correspondence Address: RATNERPRESTIA, P.O. BOX 980, VALLEY FORGE, PA, 19482, US

(21) Appl. No.: 12/532,035

(22) PCT Filed: Mar. 19, 2008
(86) PCT No.: PCT/US08/03562

§ 371 Date: May 25, 2010

Related U.S. Application Data
(60) Provisional application No. 60/896,094, filed on Mar. 21, 2007.

(51) Int. Cl. A61B 1/00

(52) U.S. Cl. 600/114

(57) ABSTRACT

An endoscopic overtube and method for achieving safe and effective endoscope passage and maintenance of pneumoperitoneum, thereby facilitating natural orifice transluminal endoscopic surgery (NOTES) in a body cavity is provided. The endoscopic overtube is designed to facilitate simultaneous use of at least two endoscopes and includes an sheath that has a proximal portion, a distal portion, and a passageway extending between the proximal portion and the distal portion. A cuff is coupled to the distal portion of the sheath and is inflatable. When the cuff is inflated, a distally facing surface of the cuff contacts an inner lumenal wall, and a proximally facing surface of the cuff contacts an outer peritoneal wall. An inflation lumen is connected to the cuff to deliver inflation fluid to inflate and/or deflate the cuff. A closure device is also provided with the endoscopic overtube for closing an incision in a tissue wall.

### CROSS REFERENCE TO RELATED APPLICATIONS

This application claims priority of U.S. provisional application Ser. No. 60/896,094, entitled NATURAL ORIFICE TRANSLUMINAL ENDOSCOPIC SURGERY OVERTUBE AND METHOD OF INTRODUCING AN ENDOSCOPE, filed Mar. 21, 2007, which is incorporated herein by reference in its entirety.

### FIELD OF THE INVENTION

The present invention relates to surgical devices, and more specifically, an endoscopic overtube for natural orifice transluminal endoscopic surgery (NOTES).

### BACKGROUND OF THE INVENTION

Similar to the advent of laparoscopy in general surgery in the late 1980's, the next major minimally invasive advance appears to be Natural Orifice Transluminal Endoscopic Surgery (NOTES). NOTES involves operations within the abdominal cavity, for example, by accessing the target site of interest through natural openings of the body (i.e., mouth, anus, vagina, urethra, etc.). Access to the abdominal cavity for appendectomy, for example, may be achieved by inserting an endoscope into the upper gastrointestinal (GI) tract via the mouth and through an internal incision in the stomach to gain access to the abdominal cavity. Compared to laparoscopic surgery, potential benefits of NOTES include “scarless” surgery, faster post operative recovery time, and lower risk of transabdominal wound infections. Thus far, there has been extensive animal work performed and patients have already successfully undergone appendectomies in India via NOTES.

Because of the challenges associated with new procedures such as NOTES, there is a need for improved apparatus and methods for introducing endoscopes for transluminal endoscopic surgery in a body cavity. The present invention addresses this need, among others.

### SUMMARY OF THE INVENTION

In one aspect, the invention provides an endoscopic overtube that is configured to receive at least one endoscope for endoscopic surgery. The endoscopic overtube includes an elongate sheath that is configured to be introduced through a naturally occurring body orifice, and the sheath includes a proximal portion, a distal portion, and a passageway extending between the proximal portion and the distal portion. A cuff is coupled to the distal portion of the sheath and has an inflated and a deflated condition. When the cuff is in the inflated condition, a distally facing surface of the cuff is positioned to contact the inner lumenal wall of the organ being traversed to gain entrance into a body cavity, and a proximally facing surface of the cuff is located distal of the distally facing surface and positioned to contact an outer peritoneal wall of the organ being traversed. An inflation lumen is connected to the cuff to deliver inflation liquid, gas, or foam to inflate and/or deflate the cuff.

In another aspect, an endoscopic overtube includes an outer sheath that is configured to be introduced through a naturally occurring body orifice, and the outer sheath includes a proximal portion, a distal portion, and a passageway extending between the proximal portion and the distal portion. An inner sheath is configured to be introduced within the passageway of the outer sheath, and the inner sheath also includes a proximal portion, a distal portion, and a passageway extending between the proximal portion and the distal portion. A cuff is coupled to each of the distal portion of the inner and outer sheaths, and each cuff has an inflated condition and deflated condition. When the cuff of the outer sheath is in the inflated condition, a distally facing surface of the cuff is positioned to contact an inner lumenal wall. When the cuff of the inner sheath is in the inflated condition, a proximally facing surface of the cuff is positioned to contact an outer peritoneal wall of the organ traversed to gain entry to a body cavity. An inflation lumen is connected to each cuff of the inner and outer sheaths to deliver inflation fluid such as liquid, gas, or foam to inflate and/or deflate each cuff.

In yet another aspect, a method is provided for introducing an endoscope for transluminal endoscopic surgery in a body cavity. The method includes introducing an elongate sheath through a naturally occurring body orifice and then passing a distal portion of the sheath through an incision in the wall of the organ being traversed. A cuff that is coupled to the sheath is inflated such that a distally facing surface of the cuff contacts the inner lumenal wall and a proximally facing surface of the cuff contacts the outer peritoneal wall. An endoscope is then introduced through a passageway defined by the sheath and into the body cavity.

In yet another aspect, a method is provided for introducing an endoscope for transluminal endoscopic surgery in a body cavity and includes introducing an endoscope through a lumen defined by an endoscopic overtube. The endoscope and the overtube are then introduced through a naturally occurring body orifice and into a body organ. A second endoscope is introduced through a second lumen defined by the endoscopic overtube and an incision is provided through the wall of the body organ. The distal portion of the endoscopic overtube is then guided through the incision and into the body cavity. The second endoscope may then be introduced into the body cavity through the second lumen of the endoscopic overtube.

### BRIEF DESCRIPTION OF THE DRAWINGS

The invention is best understood from the following detailed description when read in connection with the accompanying drawings, with like elements having the same reference numerals. This emphasizes that according to common practice, the various features of the drawings are not drawn to scale. On the contrary, the dimensions of the various features are arbitrarily expanded or reduced for clarity. Included in the drawings are the following figures:

FIG. 1 is a schematic anatomical view of a human body showing placement of an endoscopic overtube through a naturally occurring body orifice and into a body cavity according to an exemplary aspect of this invention;

FIG. 2 is a schematic cross sectional side view of an exemplary embodiment of an endoscopic overtube in accordance with the present invention;

FIG. 3 is a schematic cross sectional end view along the III line of the endoscopic overtube shown in FIG. 2;

FIG. 4 is a schematic cross sectional end view of another exemplary embodiment of an endoscopic overtube;

FIG. 5 is a schematic cross sectional side view of yet another exemplary embodiment of an endoscopic overtube in accordance with the present invention;

FIG. 6 is a schematic cross sectional side view of still another exemplary embodiment of an endoscopic overtube in accordance with the present invention;

FIG. 7 is a schematic cross sectional end view along the VII-VII line of the endoscopic overtube shown in FIG. 6;

FIG. 8 is a schematic cross sectional end view of a component of an exemplary embodiment of the present invention;

FIG. 9 is a schematic view of another exemplary embodiment of an endoscopic overtube according to aspects of this invention;

FIG. 10 is a schematic expanded view of a portion of the endoscopic overtube shown in FIG. 9;

FIG. 11 is a schematic side view of yet another exemplary embodiment of an endoscopic overtube according to aspects of this invention;

FIGS. 11A, 11B, 11C, 11D, and 11E are schematic cross sectional end views along the 11A, 11B, 11C, 11D, and 11E lines of the endoscopic overtube shown in FIG. 11;

FIG. 12 is a schematic view of a portion of still another exemplary embodiment an endoscopic overtube according to aspects of the present invention;

FIG. 13 is a schematic perspective view of an exemplary embodiment of a mouthpiece component according to an aspect of the present invention;

FIGS. 14A, 14B, 14C, and 14D are schematic views of a sequence of steps for introducing an endoscopic overtube and endoscopes into a body cavity in an exemplary method according to an aspect of this invention;

FIG. 15 is a schematic view of a portion of still another exemplary embodiment an endoscopic overtube according to aspects of the present invention;

FIGS. 16A, 16B, and 16C are schematic views of a sequence of steps for closing an incision of an organ in an exemplary method according to an aspect of this invention;

FIG. 17 is schematic plan view of an exemplary embodiment of a component of an overtube assembly according to an aspect of the present invention;

FIGS. 18A, 18B, and 18C are schematic views of a portion of yet another exemplary embodiment of an endoscopic overtube according to an aspect of this invention;

FIGS. 19A and 19B are schematic views of still another exemplary embodiment of an endoscopic overtube according to an aspect of this invention; and

FIGS. 20A, 20B, and 20C are schematic views of a portion of yet another exemplary embodiment of an endoscopic overtube according to an aspect of this invention.

### DETAILED DESCRIPTION OF THE INVENTION

Aspects of the invention will now be described with reference to the figures. Such figures are intended to be illustrative rather than limiting and are included herewith to facilitate the explanation of the present invention.

The principle that catapulted laparoscopy into an accepted surgical technique beyond conventional “open” surgery was the ability to perform the same operation as in the open technique, but using a minimally invasive approach. In addition to achieving a safe and effective method for endoscope passage and maintenance of pneumoperitoneum, exemplary embodiments of this invention similarly provide a safe technique for allowing two surgeons to operate, simultaneously, using two endoscopes. Using two endoscopes simultaneously will allow the surgeon to employ the necessary techniques that are the central principles of surgery, multi-angle visual perspective and the capacity to retract, expose and provide traction and countertraction at surgical sites. The ability to use two endoscopes simultaneously will be beneficial for NOTES to help fulfill the criterion that the surgeon be able to perform an uncompromised operation, when compared to an open or laparoscopic approach.

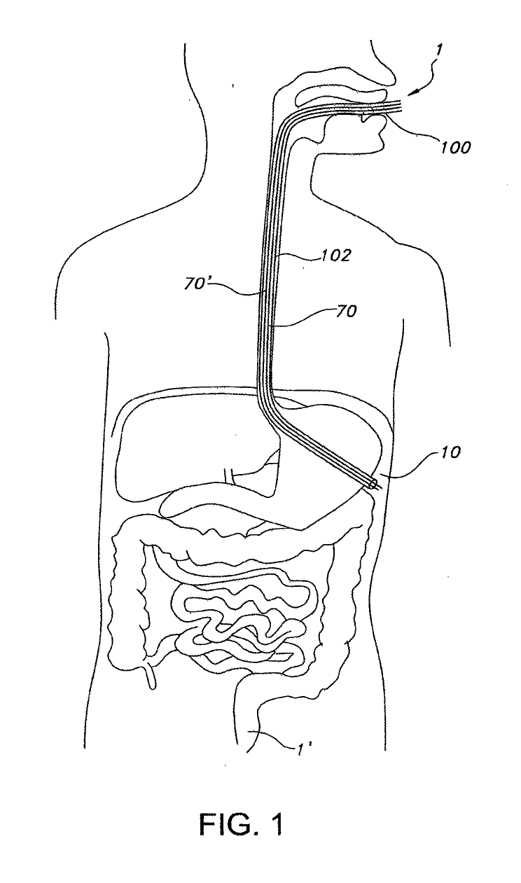

Referring generally to the figures (FIGS. 1-20), in accordance with an exemplary embodiment, the invention provides an endoscopic overtube 100, 200, 300, 900, 1100, 1200, 1400, 1500, 1800, 1900, 2000 configured to receive at least one endoscope 70, 70′ for endoscopic surgery. The endoscopic overtube 100, 200, 300, 900, 1100, 1200, 1400, 1500, 1800, 1900, 2000 includes a sheath 102, 202, 302, 302′, 902, 1102, 1202, 1402, 1502, 1802, 1902, 2002 that is configured to be introduced through a naturally occurring body orifice 1, 1′, for example, and the sheath 102, 202, 302, 302′, 902, 1102, 1202, 1402, 1502, 1802, 1902, 2002 includes a proximal portion 2, a distal portion 4, and a passageway 3, 3′ extending between the proximal portion 2 and the distal portion 4. A cuff 104, 204, 204′, 304, 304′, 904, 1104, 1104′, 1404, 1804, 1904, 2004 is coupled to the distal portion 2 of the sheath 102, 202, 302, 302′, 902, 1102, 1402, 1802, 1902, 2002 and has an inflated condition and a deflated condition. When the cuff 104, 204, 204′, 304, 304′, 904, 1104, 1104′, 1404, 1804, 1904, 2004 is in the inflated condition, a distally facing surface 121, 221, 321, 921, 1421 of the cuff is positioned to contact an inner lumenal wall 51, and a proximally facing surface 122, 222, 322, 922, 1422 of the cuff is located distal of the distally facing surface 121, 221, 321, 921, 1421 and positioned to contact an outer peritoneal wall 52. An inflation lumen 106, 1106a, 1106b is connected to the cuff 104, 204, 204′, 304, 304′, 904, 1104, 1104′, 1404 to deliver inflation fluid such as liquid, gas, or foam to inflate and/or deflate the cuff 104, 204, 204′, 304, 304′, 904, 1104, 1104′, 1404.

In a further embodiment of the present invention, a method for introducing an endoscope 70, 70′ for transluminal endoscopic surgery in a body cavity 10, 60 is provided. The method includes the step of introducing an elongate sheath 102, 202, 302, 302′, 902, 1102, 1202, 1402, 1502, 1802, 1902, 2002 through a naturally occurring body orifice 1, 1′ and passing a distal portion 4 of the sheath 102, 202, 302, 302′, 902, 1102, 1202, 1402, 1502, 1802, 1902, 2002 through an incision 30 in the wall 50 of the organ being traversed. A cuff 104, 204, 204′, 304, 304′, 904, 1104, 1104′, 1404, 1804, 1904, 2004 that is coupled to the sheath 102, 202, 302, 302′, 902, 1102, 1402, 1802, 1902, 2002 is inflated such that a distally facing surface 121, 221, 321, 921, 1421 of the cuff contacts the inner lumenal wall 51, and a proximally facing surface 122, 222, 322, 922, 1422 of the cuff contacts the outer peritoneal wall 52. The endoscope 70, 70′ is then introduced through a passageway 3, 3′ defined by the sheath 102, 202, 302, 302′, 902, 1102, 1202, 1402, 1502, 1802, 1902, 2002 and into the body cavity 10, 60.

Referring now to the individual figures in detail, FIG. 1 illustrates schematically an anatomical view of a human body having an endoscopic overtube 100 introduced through a naturally occurring body orifice and into a body cavity 10 for endoscopic surgery. The naturally occurring body orifice includes, but is not limited to, the upper GI tract 1 (via the mouth), lower GI tract 1′ (via the anus), urethra, and vagina.

When endoscopic surgery in a body cavity 10 is performed from the upper GI tract 1 via the mouth, a medical mouthpiece assembly (FIG. 13, 1300)) may be provided to hold the mouth open and introduce the endoscopic overtube 100 into the upper GI tract 1. The medical mouthpiece may be integral with or separate from the endoscopic overtube 100 and may include a mouth guard and a band that fixes around a patient's head. The mouthpiece assembly may also include adjustable clamps that retain one or more endoscopes 70, 70′ at specific points in space and free a surgeon's hands from having to hold the endoscopes 70, 70′ in place. In yet another exemplary embodiment the mouthpiece assembly may include a clamp for an endotracheal tube. Additional features and aspects of exemplary medical mouthpiece assemblies are described in U.S. Pat. Nos. 5,533,523 and 6,517,549, which are incorporated fully herein by reference.

In an exemplary embodiment, an endoscope 70, 70′ may be first inserted through an opening in the mouthpiece assembly and the overtube 100 is then fed over the endoscope 70, 70′, using the endoscope 70, 70′ as a guide. In another embodiment, after the endoscopic overtube 100, has been introduced through the mouthpiece assembly and within the naturally occurring body orifice, endoscopes 70, 70′ may be introduced within a passageway of the endoscopic overtube 100. A typical endoscope 70, 70′, such as a gastroscope, includes a flexible tube having a distal end with a light guide, a camera, and a working outlet, for example, although any number and type of ports can be provided. The light guide illuminates the area in front of the distal end of the endoscope and images are relayed by the camera to a monitor or an eyepiece. Endoscopic instruments such as biopsy forceps, laser probes, and the like, can be passed in and out of the working outlet or port for use during the endoscopic surgical procedure. Additional features and aspects of exemplary endoscopes are described in U.S. Pat. Nos. 6,997,931, 6,918,871 and 5,846,182 which are incorporated fully herein by reference.

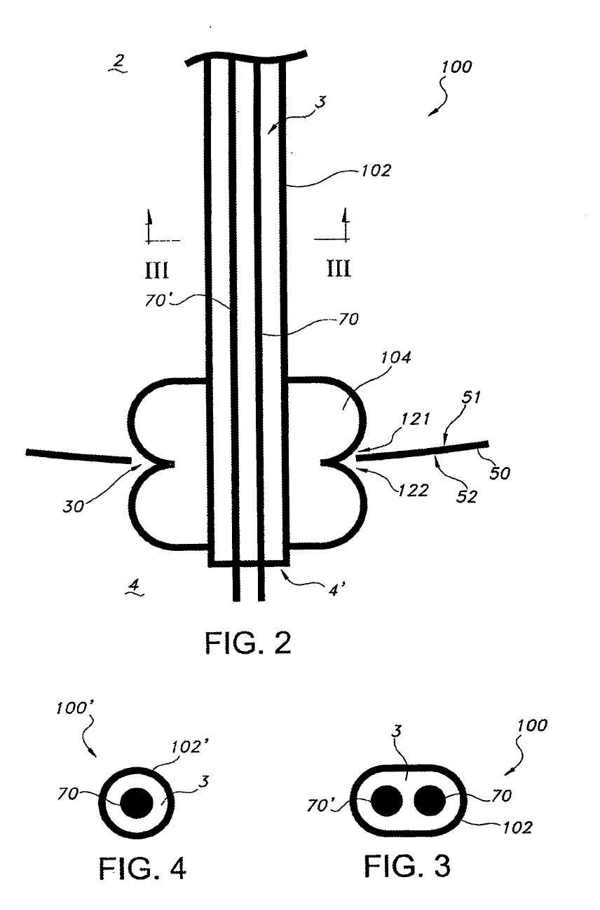

FIG. 2 illustrates a schematic side view of an exemplary embodiment of the endoscopic overtube 100 in accordance with the present invention. The overtube 100 includes a sheath 102 that is configured to be introduced into the naturally occurring body orifice. The sheath 102 can be flexible or rigid and can be made of any suitable medical grade material such as silicone, PVC, or PTFE, for example. The sheath 102 includes a proximal portion 2 and a distal portion 4 such that when the sheath 102 is introduced into the naturally occurring body orifice, the proximal portion 2 is adjacent the body orifice opening and the distal portion 4 extends away from the body orifice opening. A passageway 3 extends between the proximal portion 2 and the distal portion 4 and is configured to receive one or more (two endoscopes 70, 70′ being shown in FIG. 2). In an exemplary embodiment, the sheath 102 has a fixed length to accommodate natural body orifices of a certain length or size. Alternatively, the sheath 102 may have an adjustable length to accommodate body orifices of various lengths and sizes.

The distal portion 4 of the sheath 102 includes a cuff 104 that is inflatable/deflatable. The cuff 104 surrounds the sheath 102 and may be made of an elastic material such as rubber, latex, or a like material. The cuff 104 may be connected to the sheath 102 by conventional fastening techniques known in the art, such as adhesives or vulcanization for example. In an exemplary embodiment, the cuff 104 may be a Fogarty-style balloon which smoothly hugs the sheath 102 when the balloon is deflated so that deflated balloon material does not gather and bunch up. Additional features and aspects of a Fogarty-style balloon are described in U.S. Pat. No. 3,435,826, which is incorporated fully herein by reference. In yet another embodiment, the cuff 104 of endoscopic overtube 100 may be similar to a Foley-style catheter as described in U.S. Pat. No. 6,254,570, which is incorporated fully herein by reference.

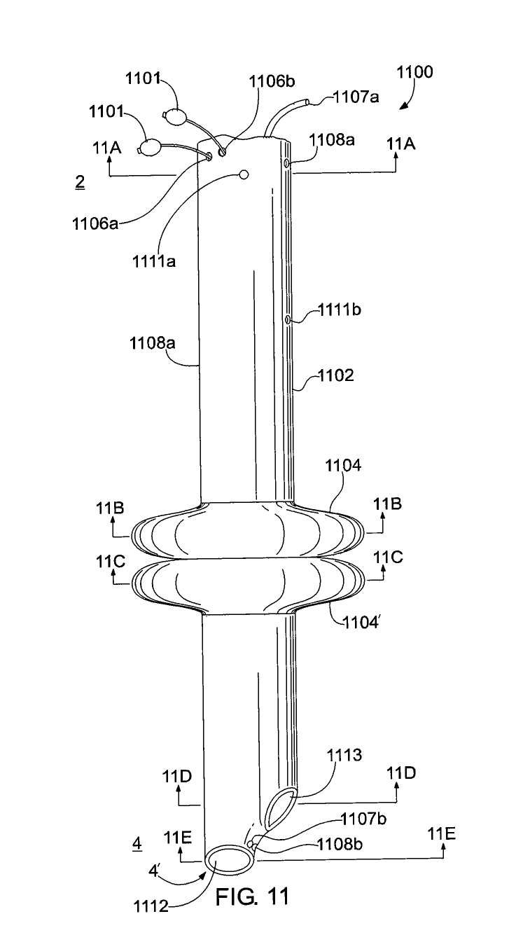

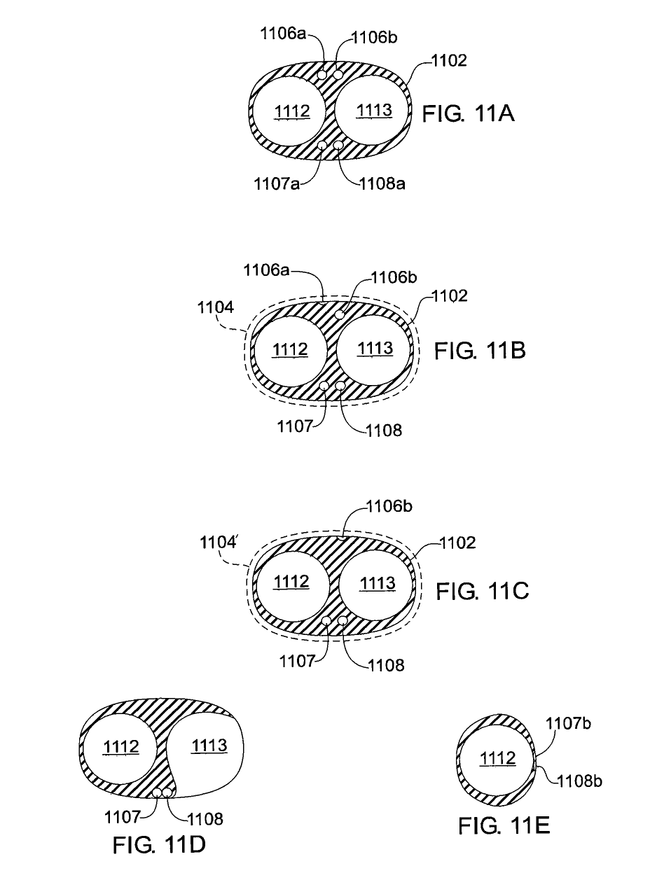

The endoscopic overtube 100 includes an inflation lumen (see for example inflation lumen 106, FIG. 8) that is in fluid communication with the cuff 104 to inflate and/or deflate the cuff 104. The inflation lumen, for example, may be connectable to a syringe (not shown) which supplies fluid/vacuum to inflate and/or deflate the cuff 104. In an exemplary embodiment, the endoscopic overtube 100 includes an insufflation lumen (see for example insufflation lumen 1107b, FIG. 11) which provides insufflation fluid such as CO₂ to inflate a body cavity 10 (FIG. 1). The cuff 104 can also be filled with a self expanding/conforming foam where the natural state is for it to be inflated and the purpose of the cuff-accessing lumen is to remove air from the cuff for inflation, thereby collapsing it, and when the lumen is open, air is drawn in allowing the cuff to self-inflate. Additional air could be added, in this configuration, to further inflate the cuff. Additional features and aspects of examples of structures that can be used to form the cuff, inflation lumen, and insufflation lumen, are described in U.S. Pat. Nos. 4,584,998, 5,620,408, and 5,443,064, which are incorporated fully herein by reference.

When the sheath 102 is initially introduced into the naturally occurring body orifice, cuff 104 may be deflated or inflated. After the distal end 4′ of the sheath 102 passes through an opening 30 in the wall of the organ being traversed 50 (such as through an incision in the tissue wall 50 of a stomach, colon, etc.), cuff 104 may be inflated or deflated. The cuff 104 is shaped such that when it is inflated, a distally facing surface 121 of the cuff 104 faces distally such it can contact or contacts the inner lumenal wall 51, and a proximally facing surface 122 faces proximally to contact the outer peritoneal wall 52.

Thus, although gaps are shown in schematic FIG. 2, a partial or complete seal may be formed around the opening 30 of the wall 50 of the organ being traversed and prevent or reduce leakage through the opening 30 in the wall 50 of the organ. For endoscopic surgery through the upper GI tract, for example, gastric fluid from the stomach would be prevented from entering the body cavity 10 (FIG. 1). Also, the flow of insufflation gas from the body cavity 10 and into stomach, for example, is prevented, reduced or inhibited.

In an exemplary embodiment, the pressure between the distally facing surface 121 and the proximally facing surface 122 of the cuff 104 is sufficient to prevent or reduce leakage through the opening 30 in the wall 50 of the organ while also preventing necrosis of the portion of the wall 50 of the organ between the distally facing surface 121 and the proximally facing surface 122. It will be appreciated that the inflation pressure of the cuff 104 can be adjusted to tighten or loosen the contact with, or engagement of, the wall 50 of the organ.

FIG. 3 illustrates a cross sectional view of the endoscopic overtube 100 shown in FIG. 2 along the line. The sheath 102 is generally ovoid in cross section to provide alignment of the endoscopes 70, 70′ in the passageway 3 of the sheath 102. The use of two or more endoscopes, as illustrated schematically in FIG. 3, is beneficial to overcome the limited vision associated with the use of a single endoscope and the difficulty in perceiving distances between instruments (e.g., biopsy forceps, laser probes, etc.) that are passed through a single endoscope. Nevertheless, passing two endoscopes through a natural orifice such as the esophagus, for example, can be both difficult and potentially dangerous. Even if the endoscopes can be successfully passed into the stomach, a potentially dangerous situation would exist if one endoscope bowed out and pressed against the other endoscope. In this situation, the endoscopes could tear the esophagus.

In the embodiment illustrated in FIG. 2, however, the sheath 102 reduces or prevents injury to the esophagus or other body orifice. For this reason, sheath 102 is preferably flexible yet not distensible.

The use of two endoscopes as illustrated in FIG. 3 would allow two surgeons or a surgeon and an assistant to work together and provide increased visual perspective of the operative area. Furthermore, the use of two endoscopes 70, 70′ permits the ability to approach an operative target from different directions.

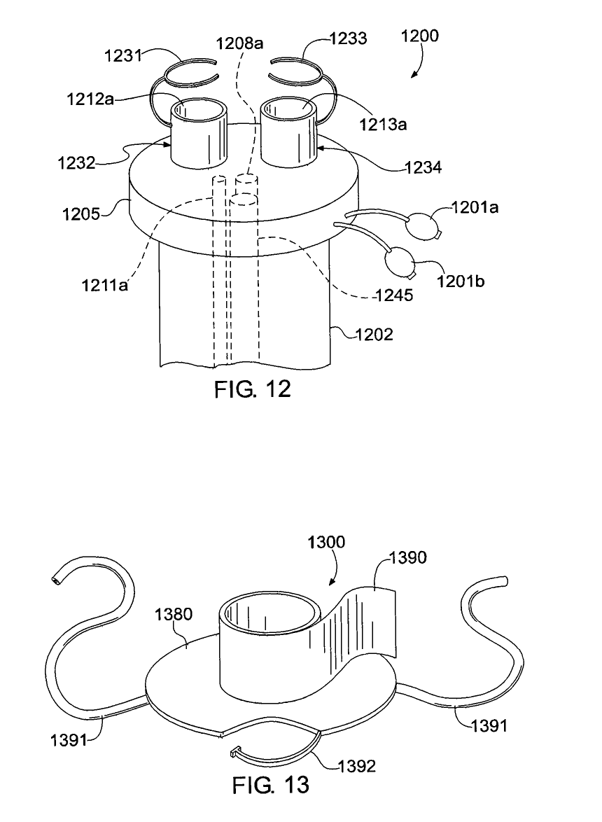

In an exemplary embodiment, the sheath 102 may be sized to closely or even snuggly fit the endoscopes 70, 70′ within the sheath 102 such that the outer circumference of each of the endoscopes 70, 70′ contacts or nearly contacts each other and contacts or nearly contacts the inner surface of the sheath 102. For example, if two endoscopes were placed side by side in an oval overtube 100 that is introduced within the esophagus, compared to a circular overtube, the cross sectional area would decrease by approximately 40% and the distended circumference of the esophagus would decrease by approximately 20%. In an alternative embodiment, an overtube may include gaskets that are configured to receive each endoscope (see for example gaskets 1212a, 1213a in FIG. 12). The gaskets may be made of a rigid or flexible material and may have circumferences that are sized larger or equal to than the circumference of each endoscope 70, 70′.

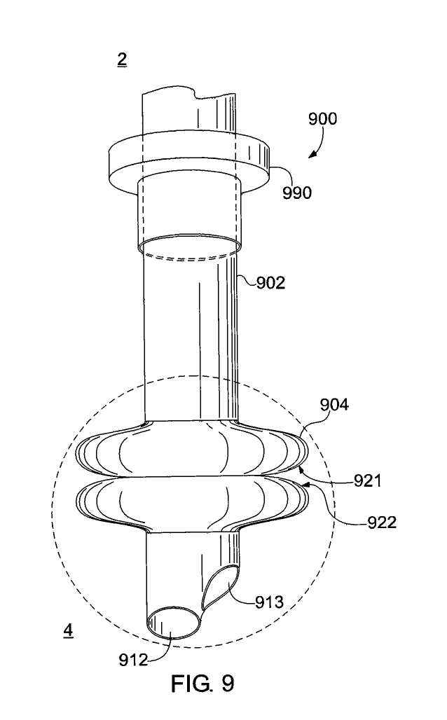

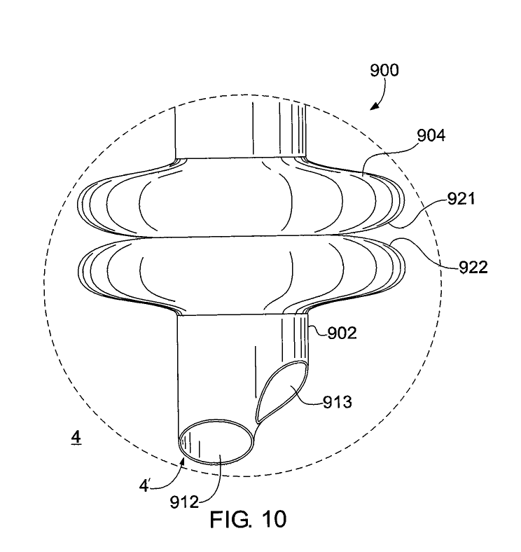

In the embodiment illustrated in FIG. 3, the sheath 102 of the overtube 100 includes a single lumen for the endoscopes 70, 70′. Alternatively, in a preferred embodiment, the sheath 102 of the overtube 100 may include a separate or partially separated lumen for each endoscope 70, 70′ (see for example lumens 912, 913 in FIGS. 9 and 10). The sheath 102 may be constructed such that one lumen for the second endoscope 70′ is collapsed and the other lumen fits over the first endoscope 70. Once the first endoscope 70 is introduced through the first lumen, the second endoscope 70′ could be inserted through the second lumen of the sheath 102.

FIG. 4 illustrates a cross sectional view of an alternative embodiment of an endoscopic overtube 100′. As described above in FIGS. 2 and 3, the endoscopic overtube 100′ includes a sheath 102′ that is configured to be introduced into a naturally occurring body orifice. The sheath 102′ has a circular cross sectional shape that may be designed to fit within a similarly shaped body orifice lumen. The passageway 3 of sheath 102′ which receives an endoscope 70 may have a cross sectional area this is greater than or substantially equal to the cross sectional area of the endoscope 70′.

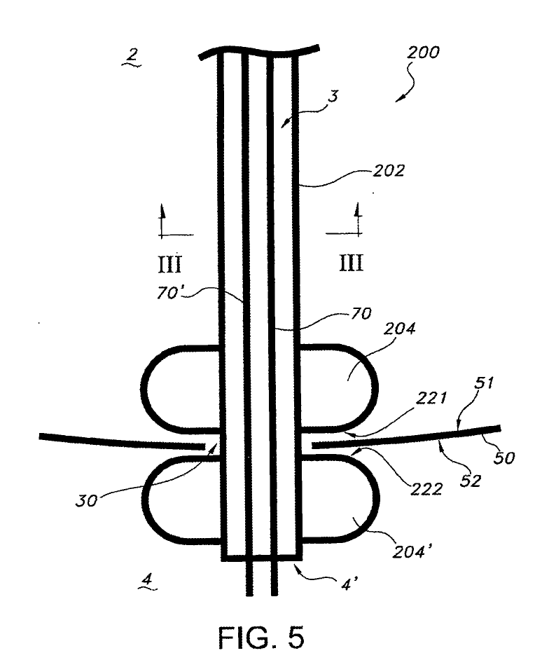

FIG. 5 illustrates a side view of an endoscopic overtube 200 according to an alternative embodiment of the present invention. As described above in connection with FIG. 2, the overtube 200 includes a sheath 202 having a proximal portion 2 and a distal portion 4. The sheath 202 is defined by a passageway 3 that extends between the proximal portion 2 and the distal portion 4. The overtube 200 includes an upper or proximal cuff 204 and lower or distal cuff 204′ coupled to the distal portion 4 of the sheath 102.

The operation of the endoscopic overtube 200 illustrated in FIG. 5 will now be described with reference the proximal cuff 204 and distal cuff 204′. After the distal end 4′ of the sheath 102 passes through an opening or incision 30 in a wall 50 of a organ to be traversed (such as through an incision in the wall 50 of a stomach, colon, etc.), proximal cuff 204 and distal cuff 204′ are inflated. In the inflated configuration, the distally facing surface 221 of the proximal cuff 204 is positioned to contact the inner lumenal wall 51, and the proximally facing surface 222 of the distal cuff 204′ is positioned to contact the outer peritoneal wall 52.

When the proximal cuff 204 and distal cuff 204′ are inflated, a seal is at least partially formed around the opening 30 of the wall 50 of the organ to be traversed to prevent leakage through the opening 30. Though gaps are shown schematically in FIG. 5, the cuffs 204, 204′ are preferably positioned at a distance from one another, or are positioned adjacent one another, such that when inflated they substantially close upon the wall 50. In an exemplary embodiment, the proximal cuff 204 and distal cuff 204′ of the endoscopic overtube 200 may be formed from materials such as those used in a Sengstaken-Blakemore tube as described in U.S. Pat. No. 4,522,205, which is incorporated fully herein by reference.

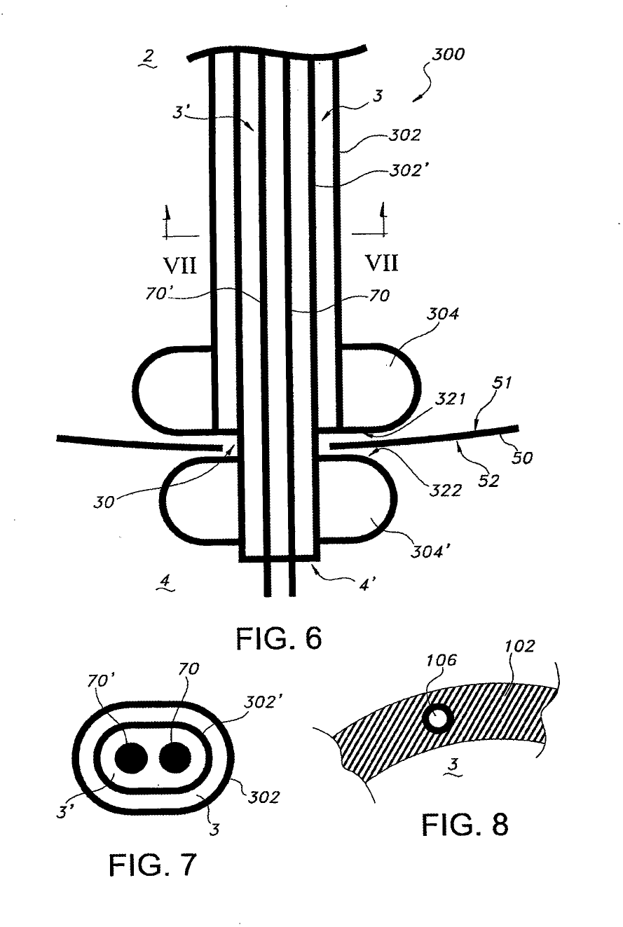

FIG. 6 illustrates schematically a side view of an alternative embodiment of the endoscopic overtube 300. The overtube 300 includes an outer sheath 302 that has a proximal portion 2 and a distal portion 4. The outer sheath 302 is defined by a passageway 3 that extends between the proximal portion 2 and the distal portion 4. The endoscopic overtube 300 also includes an inner sheath 302′ that is configured to be introduced through the passageway 3 of the outer sheath 302. The inner sheath 302′ has a proximal portion 2 and a distal portion 4. The inner sheath 302′ defines a passageway 3′ extending between the proximal portion 2 and distal portion 4 through which endoscopes 70, 70′ are introduced.

The operation of the endoscopic overtube 300 illustrated in FIG. 6 will now be described with reference to the outer sheath 302 and inner sheath 302′. First, the outer sheath 302 may be introduced into a naturally occurring body orifice such that the distal end 4′ of the outer sheath is adjacent an inner lumenal wall 51. A cuff 304 on the outer sheath may then be inflated such that a distally facing surface 321 of the cuff 304 contacts the inner lumenal wall 51. An inner sheath 302′ is introduced through the passageway 3 of the outer sheath 302 and endoscopes 70, 70′ are introduced through the passageway 3′ of the inner sheath 302′. A working instrument such as a biopsy forceps may be extended from an endoscope 70, 70′ to provide an incision in the wall 50 of the organ to be traversed. Next, the distal end 4′ of the inner sheath is passed through the incision and a cuff 304′ on the inner sheath 302′ is inflated such that a proximal facing surface 322 of the cuff 304′ contacts the outer peritoneal wall 52.

The inner sheath 302′ may be movable with respect to the outer sheath 302 such that pressure is applied to the inner lumenal wall 51 and outer peritoneal wall 52 to prevent leakage through the opening in the wall 50. More specifically, the embodiment illustrated in FIG. 6 permits relative axial movement of the sheathes 302, 302′ with respect to one another so that the pressure or force on tissue such as wall 50 can be adjusted by the surgeon. The inner sheath 302′ and outer sheath 302 may then be locked in postion with respect to each other so that additional axial movement of the sheaths is prevented. Accordingly, the surgeon can maintain an appropriate pressure preferably greater than that needed to form at least a partial seal yet less than that pressure that would cause necrosis of the tissue.

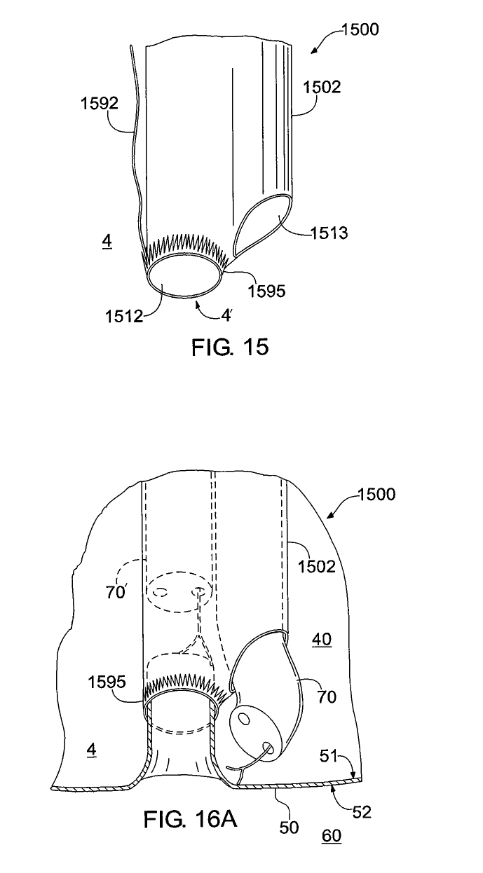

In an embodiment of the endoscopic overtube 300, the outer sheath 302 may include a closure device (1595 ,FIG. 17) coupled to the distal end 4′ of the outer sheath 304. The closing device may be utilized to close the opening 30 the tissue wall 50 after a surgical procedure has been completed. Additional aspects of the closure device are described in detail below with reference to FIGS. 15, 16A through 16C, and 17.

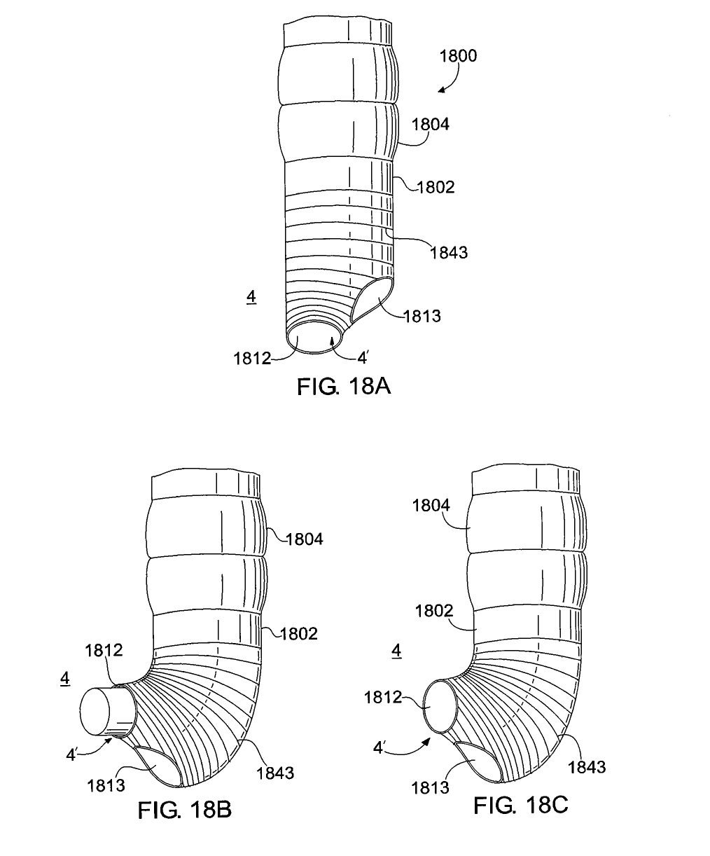

In yet another embodiment, the distal end 4′ of the inner sheath 302′ may be moveable in a radial direction (e.g., laterally) so as to permit selective orientation of the distal tip of the sheath of the overtube. Such orientation is optionally facilitated by the mechanisms used in endoscopes themselves. Directional control of the end of the sheath provides optional control of the sheath in the body cavity and can help reorient the distal portions of the endoscopes 70, 70′ and/or distal portion 4′ of the inner sheath 302′ to orient the distal end 4′ in different directions. Additional aspects and embodiments of directional control of the overtube are described in further detail with reference to FIGS. 18 through 20 below.

FIG. 7 illustrates a schematic cross sectional view of the endoscopic overtube 300 shown in FIG. 6 along the VII-VII line. As described above in FIG. 4, the outer sheath 102 and inner sheath 302′ are generally ovoid in cross section to provide alignment of the endoscopes 70, 70′ in the passageway 3′ of the inner sheath 302′. In addition, the ovoid cross section may provide better fit within the passageway of a natural body orifice (such as the esophagus) or help maintain the relative orientation or rotational orientation of the inner and outer sheathes 302, 302′. In an embodiment of the endoscopic overtube 300, the inner sheath 302′ and/or outer sheath 302 may include an insufflation lumen (not shown) to provide insufflation fluid into a body cavity 10 (FIG. 1).

FIG. 8 illustrates a cross sectional view of an arcuate portion of a sheath of the endoscopic overtube 100 of FIG. 2 according to an exemplary embodiment. The sheath 102 includes or defines an inflation lumen 106 within the cross-section of the sheath 102 to provide inflation fluid such as gas or liquid to inflate a cuff on the sheath 102. In an alternative embodiment, the inflation lumen 106 may be a tube contained within or outside the passageway 3 of the sheath 102. Such a lumen (or plural lumens) is optionally provided in any of the sheathes 102, 202, 302, 302′ and is optionally provided in any shape or configuration.

Another embodiment of an endoscopic overtube, generally referred to by the numeral “900”, is illustrated in FIGS. 9 and 10. Referring first to FIG. 9, endoscopic overtube 900 includes a sheath 902 having at least one cuff 904 coupled to the sheath 902. As with prior embodiments, the cuff 904 of endoscopic overtube 900 includes a distally facing surface 921 and proximally facing surface 922, which proximally facing surface 922 is located distal of the distally facing surface 921. Endoscopic overtube 900 also includes a mouthpiece guard 990 as described above.

Endoscopic overtube 900 differs from prior embodiments in that the distal portion 4 of the endoscopic overtube 900 is uniquely configured for the delivery of a first and second endoscope through strategically positioned lumens. As would be described in further detail below, the strategic positioning of these lumens in endoscopic overtube 900 permits the insertion of an endoscope at positions relative to one another that facilitate visualization and manipulation of tissue in the body cavity. The locations of the first and second lumen of the endoscopic overtube 900 also facilitate the insertion of the sheath 902 through an incision. More specifically, the distal portion 4 of endoscopic overtube 900 includes a first lumen 912, positioned at a distal end of the distal portion 4, and a second lumen 913, positioned proximally from the first lumen 912.

Referring now to FIG. 10, which illustrates and enlarged view of the distal portion 4 of the endoscopic overtube 900, this embodiment preferably includes a tapered distal portion 4 at the location of the first and second lumens 912 and 913. Although shown schematically in FIG. 10, it will be understood that a tapered distal portion 4 helps to facilitate the advancement of the distal portion 4 of the endoscopic overtube 900 into a body cavity through an incision. Additionally, the material used to form the distal portion 4, or a portion of distal portion 4, is optionally selected to permit collapse of one or both of the lumens 912 and 913. For example, when an endoscope is positioned within the first lumen 912 (thereby substantially preventing its collapse), an empty second lumen 913 would be permitted to collapse inwardly, thereby reducing the overall profile of the distal portion 4 of the endoscopic overtube 900. A reduced profile could then facilitate insertion of the distal portion 4 through an incision.

In the event that the distal portion 4 of the endoscopic overtube 900 is made from a flexible or soft material to permit selective collapse of one or both of the lumens, the fit between the inside diameter of one or both of the lumens around an outer surface of an endoscope should preferably be somewhat snug so that the endoscope will provide some column support to the overtube during insertion and provide some structure to the overtube as well as a guide, as will be discussed later in greater detail.

Referring now to FIG. 11, another embodiment of an overtube, generally designated by the numeral 1100, is illustrated schematically. Additionally, schematic cross-sectional end views are provided in FIGS. 11A through 11E in order to illustrate an exemplary structure of the endoscopic overtube 1100 at various points along the length of the overtube 1100. Accordingly, reference will be made to FIGS. 11 and 11A through 11E in the following paragraphs.

First referring specifically to FIG. 11, like prior embodiments, this embodiment includes a sheath 1102 and associated proximal and distal cuffs 1104 and 1104′. In a distal region 4 of the overtube 1100, the terminal end 4′ of a first lumen 1112 and a second lumen 1113 are provided.

At a proximal region of endoscopic overtube 1100, this embodiment is provided with a mechanism such as one or more cuff valves 1101 which are provided in order to facilitate the inflation and/or deflation of the proximal and distal cuffs 1104, 1104′. Also provided in the proximal portion of endoscopic overtube 1100 is an insufflation adapter or coupling 1107a, which is provided in communication with an insufflation lumen 1107b that is defined along the length of the sheath 1102 of the overtube 1100. As will be appreciated, the insufflation adapter port 1107a and lumen 1107b are provided to facilitate the delivery of insufflation gas into the working area of a body cavity during a surgical procedure. The insufflation adapter port 1107a, for example, may connect to a standard insufflator tube with standard valves to provide insufflation gas through the overtube and into the body cavity. Still referring to the proximal portion of overtube 1100, an additional port 1108a is provided for communication with a working port 1108b at the distal end portion of the overtube 1100. This additional port 1108a and working port 1108b are provided to facilitate the insertion of another component into the body cavity during the surgical procedure (e.g., a light source, a tissue manipulation device, or a lumen for smoke evaluation, irrigation, etc.).

It is notable that the proximal portion of endoscopic tube 1100 also includes an access lumen 1111a to be used in connection with a sump such as a stomach sump in order to reduce the pressure in an organ or a body cavity. For example, in the case of a stomach sump, a stomach sump may be used in order to reduce the pressure or draw a vacuum on the interior of the stomach during a procedure. For example, stomach sumps such as the Argyle Salem Sump Tube are known, and nasal sump tubes such as the Bard Nasogastric Sump Tube are known. Additional features and aspects of exemplary sumps such as nasogastric tubes are described in U.S. Pat. Nos. 5,417,664 and 4,735,607, which are incorporated fully herein by reference.

The proximal portion of the endoscopic overtube 1100 includes a stomach sump port entrance 1111a such as a connector that is positioned and configured to be connected to a standard suction tube of a stomach sump (not shown). Though the stomach sump port entrance 1111a is shown only schematically in FIG. 11 for purposes of general illustration, it will be appreciated that entrance 1111a is configured for connection (preferably releasable connection) to a conventional stomach sump. Also shown in FIG. 11 is a stomach sump port end 1111b positioned distally of the entrance 1111a yet proximally of the proximal cuff 1104. It will be appreciated that a lumen (not shown) extending through the sheath 1102 couples the entrance 1111a to the port end 1111b for fluid flow communication therebetween.

The port end 1111b is preferably positioned proximally of the proximal cuff 1104 in order to ensure that the port end 1111b will be positioned within an organ such as the stomach during the surgical procedure. More specifically, if it is beneficial or desired to reduce the pressure within the organ such as the stomach during a surgical procedure, the proximal cuff 1104 will provide a substantial seal against the wall of the organ such as the stomach in cooperation with the distal cuff 1104′. The port end 1111b will then be positioned within the interior of the organ such as the stomach so that the pressure within that organ can be reduced by operation of the sump. In another embodiment, decreasing pressure within an organ such as the stomach in the setting of increased intraperitoneal pressure in the body cavity would be adequate to form a pressure differential that forms a seal against the wall of the organ. Thus, cuffs may be optionally included and/or inflated on the overtube.

Referring now to FIGS. 11A through 11E, structures along the length of sheath 1102 are illustrated schematically. Referring first to FIG. 11A, the first lumen 1112 and second lumen 1113 are shown in juxtaposition to a cuff inflation lumen 1106a, a cuff inflation lumen 1106b, the insufflation lumen extending from insufflation adaptor 1107a, and the lumen extending from the additional working port 1108a. As is schematically illustrated in FIG. 11A, the lumens 1112, 1113, 1106a, 1106b, 1107a, and 1108a are formed in the wall of the sheath 1102. Nevertheless, these lumens can alternatively be positioned external to the sheath 1102 and can also be defined within one of the first and second lumens 1112 and 1113.

Referring to FIG. 11B, the cross section through the proximal cuff 1104 illustrates that the inflation lumen 1106a communicates with an interior of the proximal cuff 1104 at that region. The remainder of the structure of the sheath 1102 remains substantially the same, however. This permits the fluid flow communication with the inflation lumen 1106a and cuff 1104 such that the cuff can be inflated and/or deflated.

Referring to FIG. 11C, the inflation lumen 1106a is terminated or blocked proximal of this cross-sectional portion of the sheath 1102. Also, the inflation lumen 1106b is shown in fluid flow communication with an interior of the distal cuff 1104′, thus allowing the inflation and/or deflation of the distal cuff 1104′. The remainder of the structure of the sheath 1102 remains substantially the same, however.

Referring now to FIG. 11D, which shows a cross sectional schematic view at the location of the terminal end of the second lumen 1113 in a distal portion 4 of the overtube 1100, the second lumen 1113 is opened to provide an outlet port for an endoscope. Also, it is noted that inflation lumen 1106b terminates proximal of the section shown in FIG. 11D.

Finally, FIG. 11E illustrates an end view at the distal tip of the overtube 1100, which shows the open distal end of the first lumen 1112, which is positioned adjacent to the open ends of the insufflation lumen 1107b and the working port 1108b. As indicated previously, the working port 1108b can be used to facilitate smoke evacuation, irrigation, or provide an access opening for a medical device, for example.

Referring now to FIG. 12, the proximal end portion of an embodiment of an endoluminal overtube 1200 is illustrated schematically. Generally, the proximal end portion of the overtube 1200 has a port assembly 1205 that defines various ports for access to the interior of the sheath 1202 of the over tube 1200. As will be understood from the following description, these ports communicate with lumens or openings in the interior of the sheath 1202, and the port assembly 1205 is intended to be positioned exterior of a patient during a surgical procedure.

Referring to the embodiment illustrated in FIG. 12, the port assembly 1205 is mounted at a proximal end of the sheath 1202. The port assembly 1205 includes a cuff inflation valve 1201a and a cuff inflation valve 1201b. These valves 1201a and 1201b provide valve access to the lumens that lead to the cuffs (not shown in this figure). The valves 1201a and 1201b are optionally standard valves to permit the inflation or deflation of the cuffs by optional connection to a syringe. The port assembly 1205 also includes a stomach sump port entrance 1211a and a working port 1208a similar to those described in prior embodiments.

As described in connection with previous embodiments, first and second lumens 1212 and 1213 are also provided for the introduction of one or two (or more) endoscopes into the interior of the sheath 1202. Associated with each of the first and second lumen entrances/gaskets 1212a, 1213a are respective endoscope clamps 1231 and 1233. These clamps 1231 and 1233 can be rotated into position about a respective hinge 1232 and 1234. When in the position to clamp an endoscope, the clamps 1231 and 1233 assist the surgeon in holding the endoscope in place. In other words, the clamps 1231 and 1233 help to inhibit the axial or longitudinal movement of the endoscopes with respect to the sheath 1202. Finally, the port assembly 1205 also includes a connector 1245 for connection to an insufflation hose which provides insufflation fluid such as CO₂ to inflate a body cavity.

It will be appreciated that the port assembly 1205 illustrated in FIG. 12 can be formed from a variety of materials using a variety of manufacturing techniques. For example, the port assembly can be formed from a biocompatible polymeric material such as medical grade silicone, PVC, or other suitable materials. Also, the port assembly 1205 can be molded or otherwise formed and coupled to the sheath 1202 by a variety of means, including adhesive, heat bonding, and other coupling methods.

FIG. 13 provides a perspective view of an embodiment of a mouthpiece assembly generally designated by the numeral 1300. The mouthpiece assembly 1300 is intended to be positioned over the sheath of an endoscopic overtube at a location distal of the port assembly such as the port assembly 1205 shown in FIG. 12. The mouthpiece assembly 1300 includes a mouth guard 1380. Exemplary mouthpieces have been described above with reference to FIG. 1.

The mouthpiece assembly 1300 also includes a band 1390 positioned to secure the mouthpiece assembly 1300 to the sheath of an overtube. As would be understood, this band 1390 can be synched in such a way that its diameter decreases until it is snug about the outer surface of the overtube. Although not shown, the band 1390 optionally includes a ratchet feature by which the band can be tightened or synched down in order to reduce the diameter of the opening of the mouthpiece assembly 1300 until it is tight. For example, the band may be pulled like a wire tie so that the length of the free end of the band increases and the diameter of the opening of the mouthpiece assembly decreases, with the ratchet maintaining the band in a tightened condition. The mouthpiece assembly 1300 also includes a strap 1391 that can be used to position the mouthpiece assembly 1300 about the head of the patient. Finally, mouthpiece assembly 1300 includes an endotracheal tube clamp 1392 so that an air tube can be coupled to the mouthpiece assembly 1300.

Accordingly, the mouthpiece assembly 1300 is configured to hold both an endotrachael tube as well as an endoscopic overtube. The endoscopic overtube is held in place by the band 1390, and the endotrachael tube is held in place using the clamp 1392 that can be swung into position by a medical professional.

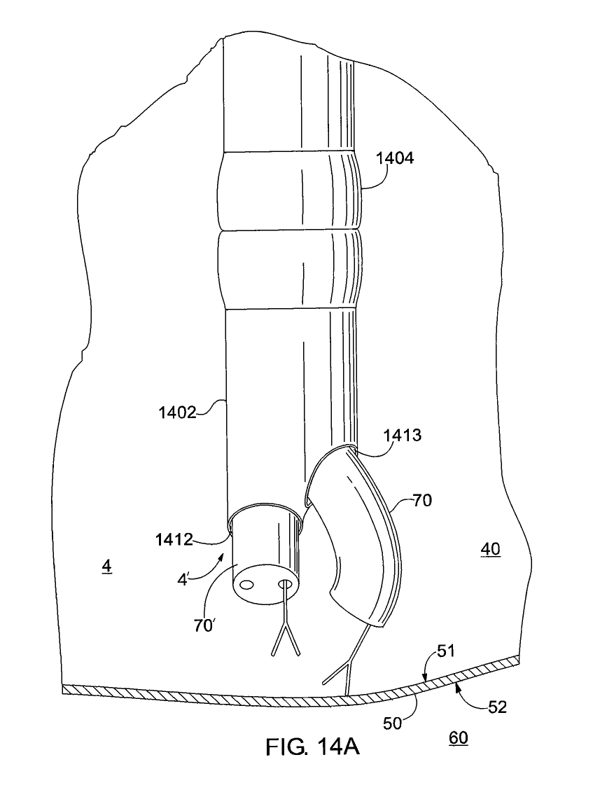

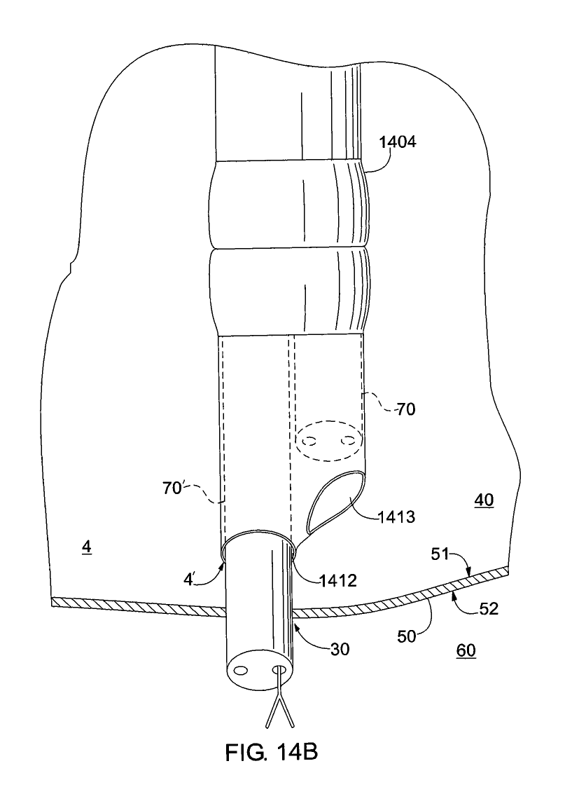

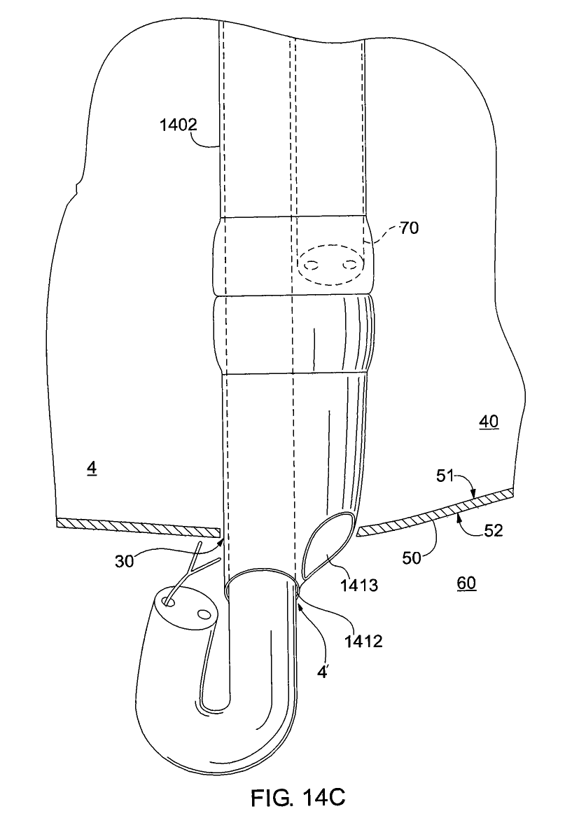

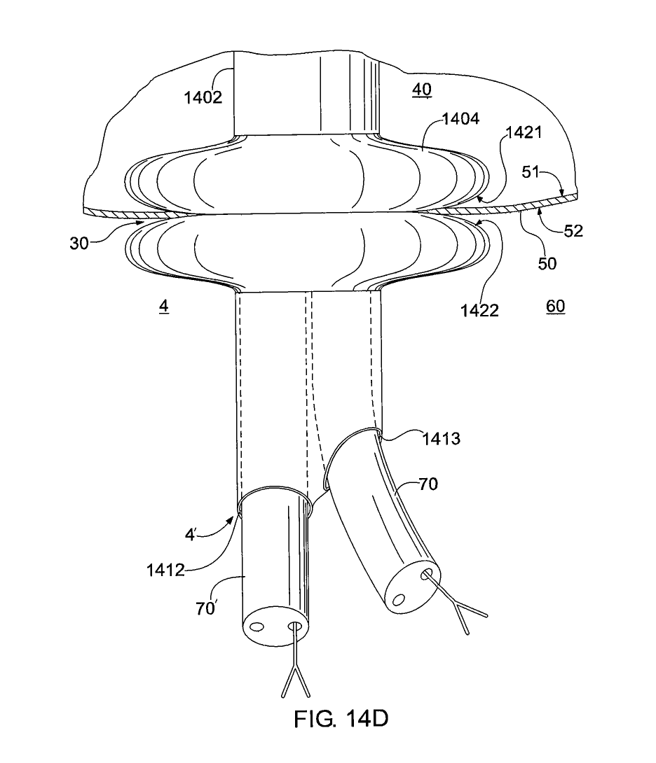

Referring now to FIGS. 14A through 14D, a method is illustrated for using the endoscopic overtube 1400, which is similar to the endoscopic overtube 900 illustrated in FIGS. 9 and 10. As with the endoscopic overtube 900, overtube 1400 includes a cuff or cuff assembly 1404 and first and second lumens 1412 and 1413. The lumens 1412 and 1413 allow the egress of distal ends of endoscopes 70 and 70′, respectively from the distal portion 4 of the sheath 1402.

In FIG. 14A, the distal portion 4 of the overtube 1400 is positioned within the stomach 40 of the patient just proximal to the wall 50 of the stomach, wherein the wall 50 divides or separates the interior of the stomach 40 from the surrounding body cavity 60. As shown in FIG. 14A, the endoscope 70 extends from the second lumen 1413 and is in a position that facilitates the making of an incision 30 in the wall 50 of the stomach 40. The endoscope 70′ extending from first lumen 1412 is positioned to visualize the actions of the endoscope 70 and/or assist with the incision by gripping the stomach wall 50. The respective procedures performed by the endoscope 70′ and 70 can be reversed depending upon the preference of the surgeon or the anatomical structures being manipulated. In other words, the endoscope 70′ can be used to grip the stomach wall 50 while the endoscope 70 makes the incision or vice versa.

Referring now to FIG. 14B, the incision has now been made in the wall 50 of the stomach 40 such that the distal tip of the endoscope 70′ can extend from the stomach through the incision in the wall 50, and into the body cavity 60. At this stage, the distal end of the endoscope 70 in lumen 1413 can be retracted (shown in FIG. 14B in broken lines). In this matter, the tapered distal end portion 4 of the overtube 1400 can then be advanced through the wall of the stomach 40 with reduced resistance. It should be noted that the endoscope 70′ first traverses the stomach wall 50 and is then followed by the distal portion of the overtube 1400 in such a way that the endoscope 70′ acts as a guide for the overtube 1400.

Referring now to FIG. 14C, it will noted that the distal tip of the overtube 1400 has now traversed the wall 50 of the stomach 40. As noted, the endoscope 70′ can be used to help manipulate the movement of the distal end 4′ of the overtube 1400. More specifically, the endoscope 70′ can be used to actually pull the overtube through the incision, if necessary. It should also be noted that the lumen 1413 (empty distal end) can collapse with the endoscope 70 retracted, thereby helping to form the tapered distal end portion 4 of the overtube 1400.

In the position illustrated in FIG. 14C, it is anticipated that at least one port/lumen of the overtube 1400 is extending into the body cavity 60. Accordingly, one or more of those ports can be utilized at this stage. For example, an insufflation port/lumen can be used to introduce insufflation gas into the body cavity. Also, a light source or tissue manipulation device can also be introduced into the body cavity through one or more ports defined in the overtube 1400. Further, in the event that a stomach sump is utilized as a component or portion of the overtube 1400 in order to evacuate the stomach or release the pressure in the stomach, such a stomach sump can be activated at the stage shown in FIG. 14C. It is anticipated that such evacuation or pressure reduction could help to facilitate a seal between the perimeter of the incision in the wall 50 of the stomach 40 and the outer surface of the sheath 1402 of the overtube 1400.

Referring to FIG. 14D, the distal end 4′ portion of the endoscopic overtube for 1400 has now been advanced farther into the body cavity 60 through the wall 50 in the stomach 40. The cuff or cuff assembly 1404 of the overtube 1400 has been inflated lumens by introducing fluids such as gas, liquid, or foam into the inflation lumens leading to the cuff or cuff assembly (1404). In this condition, the inflated cuff assembly forms a substantial seal within the incision 30 in the wall 50. More specifically, the distally facing surface 1421 of the cuff 1404 contacts an inner lumenal wall portion 51, and a proximally facing surface 1422 of the cuff 1404 contacts an outer peritoneal wall portion 52. As described above with reference to FIG. 11, in view of the pressure differential caused by the sump suction (i.e. negative pressure in the stomach, and positive pressure in the peritoneal space of the body cavity), it should be noted that the inflation of the cuff or cuffs is optional. In fact, depending on the application for the overtube 1400, the cuff or cuffs 1404 can be eliminated entirely.

As shown in FIG. 14D, the endoscopes 70′ and 70 are both extending into the body cavity 60 and extending from the first and second lumens 1412 and 1413 so that a surgical procedure can be conducted within the body cavity 60. Because of the positioning of endoscopes 70′ and 70 within the body cavity 60, it is therefore possible to visualize the procedure with adequate perspective and to conduct a surgical procedure with two endoscopes as in a laparoscopic or open operation procedure, for example.

Referring now to FIG. 15, yet another embodiment of an overtube 1500 is shown, this embodiment including a closure system 1595 configured to facilitate the closure of the incision 30 in a tissue wall 50 such as the wall of a stomach. The overtube 1500 has first and second lumens 1512 and 1513. In the length of the overtube between the distal end of the first lumen 1512 and the distal end of the second lumen 1513, a closure device 1595 (an exemplary embodiment of which is illustrated in FIG. 17) is positioned. As will be discussed later in greater detail, the closure device 1595 is preferably a “toothed” design that will be capable of engaging tissue. The device 1595 is also preferably absorbable such that the closure device 1595 may be biodegraded over time as the incision 30 in tissue 50 heals. In an exemplary embodiment, the closure device 1595 is optionally formed from a biocompatible material such as vicryl or from polyglutamic acid. Finally, a tightening band 1592 is optionally tethered to the closure device 1595 to tighten the closure device 1595 that is engaged on the tissue (such as the tissue around an incision opening 30).

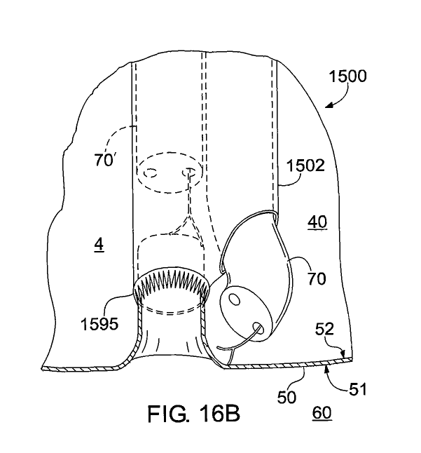

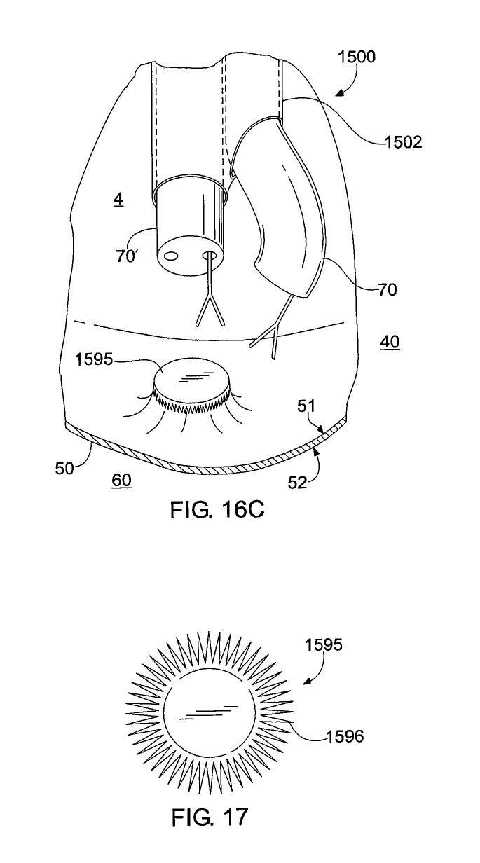

Referring to FIGS. 16A-16C, an exemplary method of closing an incision 30 in a tissue or wall such as the wall 50 of a stomach is illustrated. It will be appreciated that the overtube 1500 can be utilized as a means for introducing multiple endoscopes as well as a means for protecting the esophagus or any other natural body orifice/passageway during a NOTES procedure. Additionally, and as illustrated in FIGS. 16A-16C, the distal end portion 4 of the overtube 1500 can also be used to facilitate the closure of an incision.

As shown in FIG. 16A, the endoscope 70′ can be used to grasp the area of the incision to be closed (perhaps with the assistance of endoscope 70) and it is then pulled into the interior of the lumen 1512. This can be accomplished my grasping the tissue with endoscope 70′ and withdrawing the endoscope 70′ in an a proximal direction.

Referring to FIG. 16B, the closure device 1595 is advanced distally and off of the distal end of the overtube 1500 so that it surrounds the tissue in the area of the incision. This can be conducted with the assistance of the endoscope 70. Also, endoscope 70′, which may then release the tissue, can be used to visualize the incision area to ensure that there is an appropriate closure such that little or no leakage between the inner luminal wall 51 and the outer peritoneal wall 52 occur. Teeth of the interior surface of the closure device 1595 then act to grip the tissue and hold the incision in a closed position and also to maintain the closure device 1595 in a fixed position.

Referring now FIG. 16C, the endoscopes 70′ and 70 can be withdrawn from the area of the incision in the wall 50. The closure device 1595 then remains in position to hold the incision in a closed position. The overtube 1500 can then be withdrawn from the patient before or after the removal of the endoscopes 70′ and 70.

FIG. 17 is a schematic illustration of a closure device 1595 in a flat position. The closure device 1595 includes teeth 1596 (shown in the closed “zig-zag” configuration) that may grip the tissue wall around an incision area. The teeth 1596 may be tapered sidewardly such that the teeth extend outwardly from the center of the closure device 1596. The teeth 1596 of the closure device 1595 are configured to also bend/flip inwardly such that the teeth are oriented towards the center of the closure device 1595. In an exemplary embodiment, the closure device 1595 is compressible such that the circumference of the closure device 1595 decreases as pressure is applied. In another embodiment, the closure device 1595 may be coupled to a tightening/tensioning device (1592, FIG. 15) which may be pulled or withdrawn to apply tension and to compress the closure device 1595.

Referring now to FIGS. 18A-18C, an exemplary embodiment of an overtube 1800 with directional control of the distal portion 4 of the sheath 1802 is illustrated. As shown in FIG. 18A, the distal portion 4 of the overtube sheath 1802 includes a bendable corrugated portion 1843. The corrugated portion 1843 may include folds along the sidewall of the sheath 1802 (like a bendable drinking straw) to permit selective orientation of the distal tip 4′ of the sheath of the overtube 1800. In an exemplary embodiment, the corrugated portion 1843 may be distensible in a direction along the longitudinal length of the sheath 1802 and may rotate axially with respect to the sheath 1802.

FIG. 18B illustrates an exemplary embodiment of the operation of the corrugated portion 1843 of the sheath 1802. When an endoscope 70′ is advanced within the lumen 1812 of the sheath 1802, the endoscope 70′ may be manipulated, such that the distal end of the endoscope 70′ is oriented in a desired viewing direction. As the endoscope 70′ is manipulated, the corrugated portion 1843 of the sheath 1802 bends in the direction of the endoscope 70′. The corrugated portion 1843 may be made of a flexible material such that the distal portion 4 of the overtube 1800, as shown in FIG. 18C, retains its bended shaped when the endoscope 70′ is retracted from the lumen 1812 of the sheath 1802. Thus, an angled entry port, in the form of lumens 1812 and 1813, is provided. As described above in previous embodiments, the overtube 1800 may also include a sump port entrance and exit (1111a, 1111b, FIG. 11) that is located proximal of the overtube cuff 1804.

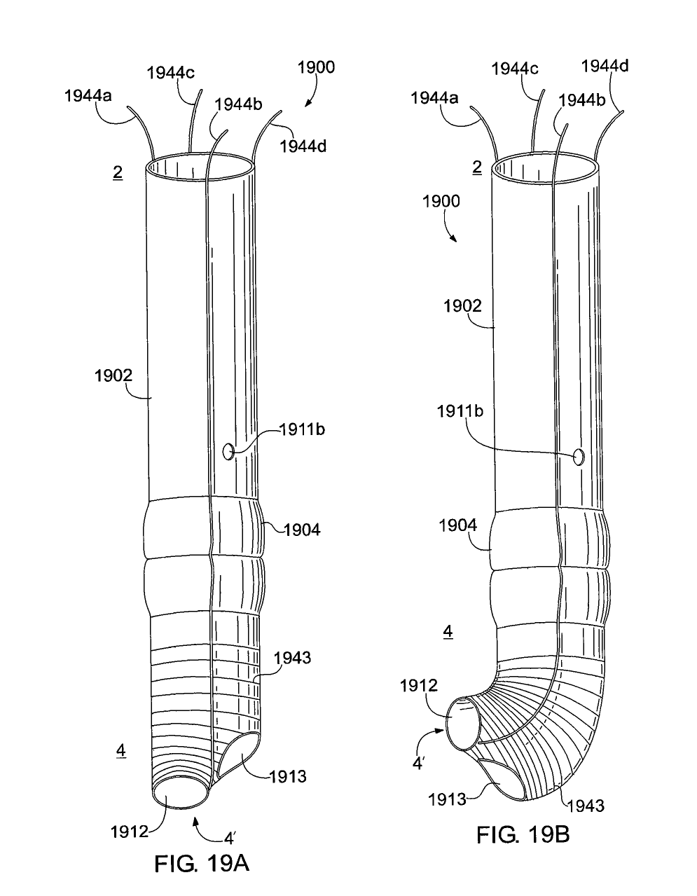

FIGS. 19A and 19B illustrate schematic views of another embodiment of an overtube 1900 having directional control of the distal portion 4 of the overtube sheath 1902. As described in previous embodiments, the overtube 1900 includes a sheath 1902 having a proximal portion 2 and a distal portion 4. The overtube 1900 also includes a sump port end 1911b that is located proximal of the overtube cuff 1904. The distal portion 4 of the sheath 1902 includes a flexible portion 1943 in which directional control is facilitated by cables 1944a-d that extend from the proximal portion 2 to the distal portion 4 of the sheath 1902. In an exemplary embodiment, the cables 1944a-d run through the wall of the sheath 1902 such as through working ports/lumens within the sheath 1902 (shown in FIG. 8) and anchor to the distal end 4′ of the overtube 1900.

In a cross sectional view of the overtube 1900, the distal portion 4 of the sheath 1902 may be divided into quadrants, with each quadrant being controlled by one or more cables. When a respective cable in a quadrant is pulled/tightened, the distal portion 4 of the sheath 1902 containing that quadrant 1943 curls sidewardly/outwardly from center the sheath 1902. As illustrated in FIG. 19B, for example, when cable 1944a is pulled, the side of the sheath 1902 adjacent cable 1944a curls outwardly, such that the distal end 4′ of the sheath 1902 is oriented in a new position. In another embodiment, when cable 1944a is released, the side of the sheath adjacent cable 1944a returns to its uncurled state. In yet another embodiment, overtube 1900 may include holders/cleats that hold the cables 1944a-d in place when then are pulled/tightened and/or released. Thus, the position of the distal end 4′ of the overtube 1900 may be maintained or manipulated during endoscopic surgery.

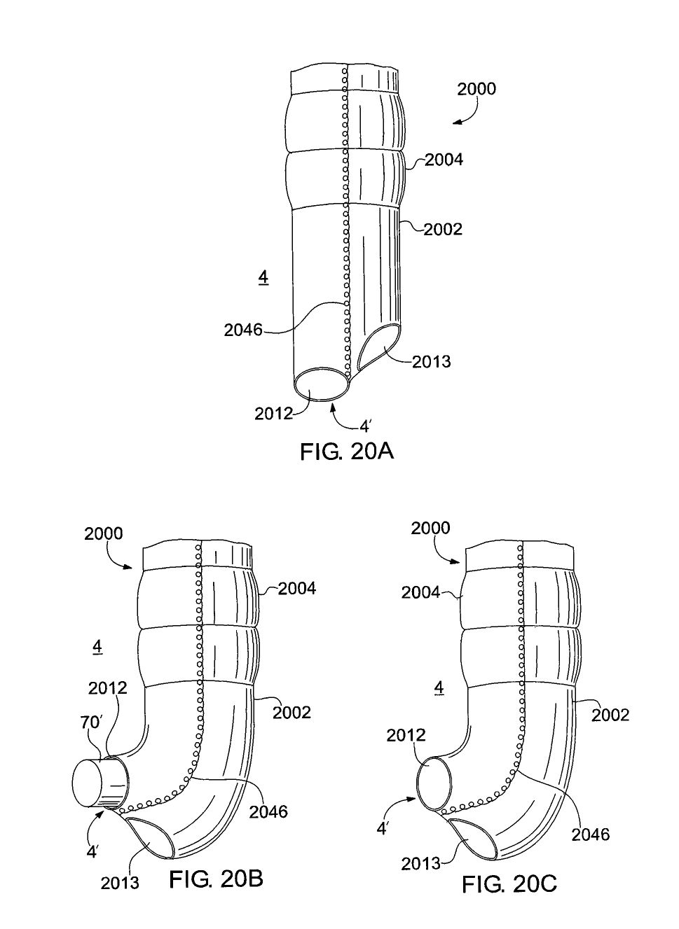

Referring now to FIGS. 20A-20C, yet another exemplary embodiment of an endoscopic overtube 2000 having directional control of the distal portion 4 of the sheath 2002 is illustrated. As shown in FIG. 20A, the overtube sheath 2002 may include one or more bendable wires 2046 within the wall of the sheath 2002. In an exemplary embodiment, the bendable wires 2046 may extend from the proximal portion of the sheath to the distal end 4′ of the sheath 2002. The bendable wire 2046 is preferably made of a material that would maintain a bend that is produced by the manipulation of the endoscope 70′ as shown in FIG. 20B. Thus, when the endoscope 70′ is retracted proximally from the distal end 4′ of the sheath 2002, as illustrated in FIG. 20C, the lumen 2012 of the sheath 2002 is oriented in a direction toward the surgical site. As described above in previous embodiments, the overtube 2000 may also include a sump port entrance and exit (1111a, 1111b, FIG. 11) that is located proximal of the overtube cuff 2004.

The embodiments described herein provide a stable platform for natural orifice translansluminal endoscopic surgery (NOTES). In particular, a first stable platform is created according to exemplary embodiments at the proximal end of the overtube because of the proximal mouthpiece clamps and proximal portion of the overtube. A second stable platform is also created internally within the body according to exemplary embodiments at the distal end of the overtube because of the distal lumens (e.g. staggered/tapered endoscope ports), inflatable cuffs, and bendable distal portion of the overtube.

The embodiments described herein also provide a technique for creating pneumoperitoneum with gas insufflation access valves at a proximal end of the overtube and an insufflation exit port for CO₂ egress at the distal tip of the overtube. A seal is created with gaskets at the proximal end of the overtube and with cuffs and/or creating a pressure differential (via sump port suction such that negative pressure exists in the stomach and pneumoperitoneum positive pressure exists outside the stomach in the body cavity) toward the distal end of the overtube. This pressure differential causes tissue to collapse around the overtube, thereby forming a seal around an incision of a tissue wall.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An endoscopic overtube configured to receive at least one endoscope for endoscopic surgery comprising:
an elongate sheath configured to be introduced through a naturally occurring s body orifice, the sheath including a proximal portion and a distal portion, and the sheath defining a passageway extending between the proximal portion and the distal portion;
a cuff coupled to the distal portion of the sheath, the cuff having an inflated condition and a deflated condition, the cuff in the inflated condition having a distally facing surface positioned to contact an inner lumenal wall and a proximally facing surface located distal of the distally facing surface and positioned to contact an outer pertioneal wall; and
an inflation lumen connected to deliver inflation fluid to the cuff to inflate and/or deflate the cuff.

2. The endoscopic overtube of claim 1, wherein the sheath is flexible.

3. The endoscopic overtube of claim 1, wherein the sheath is rigid.

4. The endoscopic overtube of claim 1, wherein the sheath has an adjustable length.

5. The endoscopic overtube of claim 1, wherein the cuff comprises a proximal cuff and a distal cuff.

6. The endoscopic overtube of claim 5, wherein the distally facing surface of the proximal cuff is positioned to contact the inner lumenal wall.

7. The endoscopic overtube of claim 5, wherein the proximally facing surface of the distal cuff is positioned to contact the outer peritoneal wall.

8. The endoscopic overtube of claim 6, wherein the inflation lumen is positioned at least partially within the sheath.

9. The endoscopic overtube of claim 1, further comprising a mouthpiece coupled to the proximal portion of the sheath.

10. The endoscopic overtube of claim 1, further comprising an insufflation lumen configured to deliver insufflation fluid into a body cavity.

11. An endoscopic overtube configured to receive at least one endoscope for endoscopic surgery comprising:
an outer sheath configured to be introduced through a naturally occurring body orifice, the outer sheath including a proximal portion and a distal portion, and the outer sheath defining a passageway extending between the proximal portion and the distal portion;
an inner sheath extending through the passageway defined by the outer sheath, the inner sheath including a proximal portion and a distal portion, and the inner sheath defining a passageway extending between the proximal portion and the distal portion;
a cuff coupled to each of the distal portion of the inner sheath and the distal portion of the outer sheath, each cuff having an inflated condition and a deflated condition, the cuff of the outer sheath in the inflated condition having a distally facing surface positioned to contact an inner lumenal wall, and the cuff of the inner sheath in the inflated condition having a proximally facing surface positioned to contact an outer peritoneal; and
an inflation lumen connected to deliver inflation fluid to each cuff of the inner sheath and the outer sheath to inflate and/or deflate each cuff.

12. The endoscopic overtube of claim 11, wherein the inner sleeve is movable with respect to the outer sheath along a longitudinal axis thereof.

13. A method for introducing an endoscope for transluminal endoscopic surgery in a body cavity, the method comprising the steps of:
introducing an elongate sheath through a naturally occurring body orifice;
passing a distal portion of the sheath through an incision in a tissue wall;
inflating a cuff coupled to the sheath such that a distally facing surface of the cuff contacts the inner lumenal wall and a proximally facing surface of the cuff contacts the outer peritoneal wall; and
introducing the endoscope through a passageway defined by the sheath and into the body cavity.

14. The method of claim 13, wherein the step of introducing the sheath comprises positioning the distal end of the sheath adjacent an inner lumenal wall.

15. The method of claim 14, wherein the step of introducing the sheath comprises introducing the endoscope through a passageway defined by the sheath to a position adjacent an inner lumenal wall.

16. The method of claim 15, wherein the step of introducing the endoscope further comprises providing an incision through the tissue wall using the endoscope, thereby providing access to the body cavity.

17. A method for introducing an endoscope for transluminal endoscopic surgery in a body cavity, the method comprising the steps of:
advancing a first endoscope through a first lumen defined by an endoscopic overtube;
advancing the endoscopic overtube through a naturally occurring body orifice into a body organ;
advancing a second endoscope through a second lumen defined by the endoscopic overtube;
providing an incision through a wall of a body organ;
advancing the first endoscope through the incision and into a body cavity;
guiding a distal portion of the endoscopic overtube through the incision and into the body cavity; and
advancing the second endoscope through the incision and into the body cavity.

18. An endoscopic overtube configured to receive at least two endoscopes for endoscopic surgery comprising an elongate sheath configured to be introduced through a naturally occurring body orifice, the sheath including at least a proximal portion and a tapered distal portion, and the sheath defining a passageway extending between the proximal portion and the tapered distal portion, wherein the tapered distal portion of the sheath defines at least two openings through which an endoscope can be extended, including a first opening located proximal of a second opening.

19. The endoscopic overtube according to claim 18, wherein the overtube includes a cuff coupled to the distal portion of the sheath, the cuff having an inflated condition and a deflated condition, the cuff in the inflated condition having a distally facing surface positioned to contact an inner lumenal wall and a proximally facing surface located distal of the distally facing surface and positioned to contact an outer peritoneal wall.

20. The endoscopic overtube according to claim 19, wherein the sheath includes a sump port located proximal of the cuff, the sump port being in communication with a sump port entrance defined in the proximal portion of the overtube.

21. The endoscopic overtube according to claim 18, further comprising a closure device associated with the tapered distal portion.

22. The endoscopic overtube according to claim 18, further comprising a means for controlling the orientation of the first opening and/or the second opening.

23. The endoscopic overtube according to claim 18, wherein the sheath comprises access ports configured to provide access to a body cavity.
